SEQUENCE LISTING (9 sequences)

SEQ ID NO: 1 - DNA, 318 bp, Artificial Sequence
  CDS (1)..(315)
  Description of Artificial Sequence Synthetic NfM-onconase gene
atgcaggattggctaacgtttcagaagaaacatatcacgaatacacgagatgtagactgcgacaatataatgtctacgaatctgtttcactgtaaggataagaatacctttatatacagtcggccagagcctgtaaaggctatctgtaaaggcattatcgcgagtaagaacgtgctgactacttccgagttctatctgtccgattgcaatgtgacttcacggccctgcaaatataagctgaagaaaagcactaacaaattttgcgtaacttgcgagaaccaggctcctgtacatttcgttggagtcgggagctgctaa

SEQ ID NO: 2 - protein, 105 aa, Artificial Sequence
  Description of Artificial Sequence Synthetic NfM-onconase amino acid sequence
MQDWLTFQKKHITNTRDVDCDNIMSTNLFHCKDKNTFIYSRPEPVKAICKGIIASKNVLTTSEFYLSDCNVTSRPCKYKLKKSTNKFCVTCENQAPVHFVGVGSC

SEQ ID NO: 3 - DNA, 139 bp, Artificial Sequence
  Description of Artificial Sequence Synthetic ONCO-N nucleotide sequence
tggctaacgtttcagaagaaacatatcacgaatacacgagatgtagactgggacaatataatgtctacgaatctgtttcactgtaaggataagaatacctttatatacagtcggccagagcctgtaaaggctatctgta

SEQ ID NO: 4 - DNA, 39 bp, Artificial Sequence
  Description of Artificial Sequence Synthetic primer
aagcttcatatgcaggattggctaacgtttcagaagaaa

SEQ ID NO: 5 - DNA, 44 bp, Artificial Sequence
  Description of Artificial Sequence Synthetic primer
cttactcgcgataatgcctttacagatagcctttacaggctctg

SEQ ID NO: 6 - DNA, 137 bp, Artificial Sequence
  Description of Artificial Sequence Synthetic ONCO-C nucleotide sequence
tgctgactacttccgagttctatctgtccgattgcaatgtgacttcacggccctgcaaatataagctgaagaaaagcactaacaaattttgcgtaacttgcgagaaccaggctcctgtacatttcgttggagtcggg

SEQ ID NO: 7 - DNA, 42 bp, Artificial Sequence
  Description of Artificial Sequence Synthetic primer
attatcgcgagtaagaacgtgctgactacttccgagttctat

SEQ ID NO: 8 - DNA, 39 bp, Artificial Sequence
  Description of Artificial Sequence Synthetic primer
ttaggatccttagcagctcccgactccaacgaaatgtac

SEQ ID NO: 9 - protein, 6 aa, Artificial Sequence
  Description of Artificial Sequence Synthetic 6xHis tag
HHHHHH

(19) United States
(12) Patent Application Publication
(10) Pub. No.: US 2005/0261170 A1
(43) Pub. Date: Nov. 24, 2005

(54) Folate conjugates and complexes

(75) Inventors: Hans J. Hansen, Picayune, MS (US); William J. McBride, Boonton, NJ (US); David M. Goldenberg, Mendham, NJ (US); Edmund A. Rossi, Nutley, NJ (US); Chien-Hsing Ken Chang, Downingtown, PA (US)

Correspondence Address: HELLER EHRMAN WHITE & MCAULIFFE LLP, 1717 RHODE ISLAND AVE, NW, WASHINGTON, DC, 20036-3001, US

(73) Assignee: Immunomedics, Inc., Morris Plains, NJ (US)

(21) Appl. No.: 11/040,114

(22) Filed: Jan. 24, 2005

Related U.S. Application Data
(60) Provisional application No. 60/538,396, filed on Jan. 22, 2004.

(51) Int. Cl. A61K 48/00; C07K 14/47; C07H 21/02

(52) U.S. Cl. 514/7; 514/44; 530/352; 536/23.1

(57) ABSTRACT

Disclosed are conjugates and complexes that include a folate receptor ligand and one or more therapeutic molecules, such as onconase or a variant thereof such as rapLR1. The conjugates and complexes may be useful as primary therapeutic agents, which may be administered with additional therapeutic or diagnostic agents. Also disclosed are kits that include the conjugates and complexes.

This application claims priority to U.S. Provisional Application No. 60/538,396, filed Jan. 22, 2004, the contents of which are hereby incorporated by reference in their entirety.

### BACKGROUND

Because the folate receptor (also called the folate binding protein, FBP) is overexpressed on certain malignant cell types, targeting of the folate receptor has been proposed as a potential mechanism for delivery of drugs and/or radiopharmaceuticals to treat cancer. See Weitman et al., (1992) Cancer Res. 52, 6708-11; Campbell et al., (1991) Cancer Res. 51, 5329-38. Folate has been conjugated to a variety of therapeutic and/or diagnostic molecules. See Mathias, et al., Bioconjugate Chem. 2000, 11, 253-257, and Wang et al., Bioconjugate Chem. 1997 September-October; 8(5):673-9 (both describing DTPA-Folate conjugates and ^{99m}Tc chelates); Siegel et al., J. Nucl. Med. 44(5):700 (2003) (describing DTPA-Folate conjugates and ¹¹¹In chelates); and Leamon et al., J. Biol. Chem. Vol. 268, No. 33, Nov. 25, 1993, pp. 24847-24854 (describing Folate-Pseudomonas Exotoxin conjugates).

Onconase and/or variants with ribonucleolytic activity, such as rapLR1, present useful therapeutic molecules for preparing folate conjugates and complexes. Onconase is a non-mammalian ribonuclease (RNase) with a molecular weight of 12,000 daltons that is purified from Rana pipiens oocytes and early embryos. Onconase causes potent inhibition of protein synthesis in rabbit reticulocyte lysate assays (IC₅₀ 10⁻¹¹ M) and when microinjected into Xenopus oocytes (IC₅₀⁻¹⁰ M). Unlike other members of the RNase A superfamily, onconase does not degrade oocyte rRNA. Upon binding to the cell surface receptors of sensitive cells and its cytosolic internalization, onconase causes cell death as a result of potent protein synthesis inhibition by a mechanism involving inactivation of cellular RNA. Onconase is not inhibited by mammalian placental ribonuclease inhibitor, which may explain onconase's enhanced cytotoxicity when compared to mammalian counterparts.

Animal toxicology studies show that onconase displays a predictable, dose-dependent and reversible toxicity in both rats (dose range 0.01-0.02 mg/kg) and dogs (0.005-0.15 mg/kg). Mice inoculated with the aggressive M109 Madison lung carcinoma and treated with both daily and weekly schedule of intraperitoneally-administered onconase, showed significantly prolonged survival. Most striking results were seen in a group of mice treated with a weekly schedule of onconase in which six of eighteen animals survived long-term and were apparently cured of cancer.

Onconase has been shown in clinical trials to have anti-tumor activity against a variety of solid tumors. In this regard it has been used both alone and combined with other anti-tumor agents such as tamoxifen, e.g., when treating patients with pancreatic cancer. When used as an anti-tumor agent, onconase can be conjugated to a marker which targets it to a specific cell type.

In a Phase I study, patients suffering from a variety of relapsing and resistant tumors were treated intravenously with onconase. A dose of 60-690 μ/m² of onconase resulted in the possible side effects of flushing, myalgias, transient dizziness, and decreased appetite in general. The observed toxicities, including the dose-limiting renal toxicity manifested by increasing proteinuria, peripheral edema, azotemia, a decreased creatinine clearance, as well as fatigue, were dose-dependent and reversible, which is in agreement with the animal toxicology studies. No clinical manifestations of a true immunological sensitization was evident, even after repeated weekly intravenous doses of onconase. The maximum tolerated dose, mainly due to renal toxicity, was found to be 960 μg/m². There were also some objective responses in non-small cell lung, esophageal, and colorectal carcinomas. Nevertheless, onconase was well-tolerated by animals and the majority of human patients tested, demonstrated a consistent and reversible clinical toxicity pattern, and did not induce most of the toxicities associated with most of the chemotherapeutic agents, such as myelosuppression and alopecia.

Onconase thus has many desirable characteristics, including small size, animal origin, and anti-tumor effects in vitro and in vivo. It is well-tolerated and refractory to human RNase inhibitors. However, onconase purified from Rana pipiens oocytes has undesirable properties. The fact that it is obtained from a natural source makes it more difficult and expensive to obtain sufficient quantities. Because it is not derived from humans, or even mammals, it typically stimulates undesirable immune responses in humans. Accordingly, it would be advantageous recombinantly to produce native onconase which retains the cytotoxic properties of onconase purified from Rana pipiens oocytes, but does not have the undesirable immune responses in humans.

Attempts to produce native onconase in E. coli by recombinant DNA methodology have failed. Onconase has an N-terminal pyroglutamyl residues which is required for proper folding of the molecule. This residue forms part of the phosphate binding pocket of onconase, and is essential for RNAse and anti-tumor activity. The initiation codon in E. coli inserts N-formyl-methionine in peptides as the N-terminal amino acid residue. Therefore, native onconase recombinantly-produced in E. coli does not have pyroglutamyl as the N-terminal residue.

WO 97/31116 discloses a method that purports to have solved the problem of producing a modified onconase that retains cytotoxic activity. It discloses a recombinant ribonuclease that has an amino terminus beginning with a methionine followed by an amino acid other than glutamic acid, a cysteine at positions 26, 40, 58, 84, 95 and 110, a lysine at position 41, and a histidine at position 119 of bovine RNAse A, and a native onconase-derived amino acid sequence. However, WO 97/31116 fails to recognize the importance of pyroglutamate as the N-terminal residue, and does not produce an onconase molecule with an N-terminal pyroglutamate. To the contrary, WO 97/31116 suggests the addition of amino terminal sequences and/or fusion at the N-terminus to a ligand molecule.

A variant of onconase called rapLR1 has been cloned from Rana Pipiens. See Chen et al., Nucl. Acids. Res., 2000 Jun. 15; 28(12):2375-82. Because onconase and variants such as rapLR1 are attractive candidates as therapeutic agents, it is desirable to create a method for targeting onconase to specific tissues. However, in addition to targeting the onconase to specific tissues, it is also important that the targeted onconase be readily internalized, in order to achieve the best therapeutic effect.

### SUMMARY

Disclosed herein are conjugates and complexes that can be targeted to and internalized by targeted tissue. The conjugates and complexes may be formulated with a pharmaceutically acceptable excipient to form a primary therapeutic agent. The conjugates and complexes include a folate receptor ligand and a ribonucleolytic moiety such as onconase or a variant such as rapLR1. The ribonucleolytic moiety may be prepared using recombinant methods and preferably has a pyroglutamyl residue at the N-terminus. Preferably, the folate conjugates and complexes retain the ribonucleolytic activity, (i.e., RNase activity), such that the conjugates and complexes are useful as therapeutic agents. Suitable folate receptor ligands include folic acid, methotrexate, and folate analogs that bind to the folate receptor. The folate receptor ligand may be directly conjugated to the ribonucleolytic moiety, or alternatively, the folate receptor ligand may be indirectly conjugated to the ribonucleolytic moiety by a linker that comprises diisocyanate, diisothiocynate, carbodiimide, bis(hydroxysuccinimide) ester, maleimide-hydroxysuccinimide ester, glutaraldehyde, or a combination thereof. In particular, the folate receptor ligand may be conjugated to the ribonucleolytic moiety at one or more lysine, histidine, or cysteine residues within the moiety.

The complexes may utilize an adapter to facilitate an interaction between the folate receptor ligand and the ribonucleolytic moiety. In one embodiment, the moiety includes a histidine tag, (including preferably at least six histidine residues and located preferably at the COOH-terminus), and as such, the moiety binds metal cations such as Ni²⁺. Similarly, the folate receptor ligand is conjugated, either directly or indirectly, to a metal-binding molecule such as a nitrilotriacetic acid residue, and as such, the ribonucleolytic moiety and the folate receptor ligand associate in a complex together with metal cations such as Ni²⁺. The folate receptor ligand and nitrilolotriacetic acid residue may be present as part of a peptide that includes additional molecules, (e.g., antigenic molecules, haptens, hard acid chelators, soft acid chelators, or combinations thereof). The peptide may be specifically bound by a multispecific binding molecule that also specifically binds a targeted tissue. As such, the complex can be targeted to the tissue.

Also disclosed is a method of treating a disease, illness, or condition comprising administering the primary therapeutic agent (i.e., the conjugates or complexes formulated with a pharmaceutically acceptable excipient), to a subject in need thereof. The primary therapeutic agent may be administered alone or with additional therapeutic and/or diagnostic agents, which may be administering before, concurrently, or after administering the primary therapeutic agent. The additional therapeutic and/or diagnostic agent may include a binding molecule (e.g., an antibody or a fragment thereof), a drug, a prodrug, a toxin, an enzyme, an enzyme-inhibitor, a nuclease, a hormone, a hormone antagonist, an immunomodulator, a cytokine, an oligonucleotide (e.g., an antisense oligonucleotide or interference RNA that targets, for example, bcl-2), a chelator, a boron compound, a photoactive agent, a radionuclide, an anti-angiogenic agent, a dye, a radioopaque material, a contrast agent, a fluorescent compound, an enhancing agent, and combinations thereof. The additional therapeutic and/or diagnostic agent may be directly associated with the primary therapeutic agent (e.g., covalently or non-covalently bound thereto).

Suitable additionally administered drugs, prodrugs, and/or toxins may include aplidin, azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, semustine streptozocin, tamoxifen, taxanes, taxol, testosterone propionate, thalidomide, thioguanine, thiotepa, teniposide, topotecan, uracil mustard, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, Pseudomonas endotoxin, or combinations thereof.

Suitable radionuclides may include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Se, ⁷⁷As, ⁸⁶Y, ⁸⁹Sr, ⁸⁹Zr, ⁹⁰Y, ⁹⁴Tc, ^{94m}Tc, ⁹⁹Mo, ^{99m}Tc, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb ²¹²Bi, ²¹²Pb, ²¹³Bi, ²²³Ra, ²²⁵Ac, or mixtures thereof. If the radionuclide is to be therapeutically, it may be desirable that the radionuclide emit 70 to 700 keV gamma particles or positrons. If the radionuclide is to be used diagnostically, it may be desirable that the radionuclide emit 25-4000 keV gamma particles and/or positrons. The radionuclide may be used to perform positron-emission tomography (PET), and the method may include performing PET.

Suitable enzymes that may be administered with the primary therapeutic agent may include carboxylesterases, glucuronidases, carboxypeptidases, beta-lactamases, phosphatases, nucleases, proteases, lipases, and mixtures thereof.

In one embodiment, a binding molecule is administered in addition to the primary therapeutic agent. The binding molecule may include an antibody or a fragment of an antibody. The binding molecule may be multivalent and/or multivalent and multispecific. In particular, the binding molecule may be bi-specific. The binding molecule may include one or more arms that specifically bind a targeted tissue and one or more that specifically bind one or more antigens present within the primary therapeutic agent. Further, the binding molecule and the primary therapeutic molecule may be utilized in a therapy that includes a “targeting” or “pre-targeting” step, as described in U.S. Ser. No. 10/150,654, U.S. Ser. No. 09/823,746, U.S. Ser. No. 09/337,756, U.S. Ser. No. 09/382,186, and U.S. 60/444,357 (filed on Jan. 31, 2003).

Where the binding molecule includes an antibody or a fragment thereof, the antibody or fragment thereof may include a human, chimeric, or humanized antibody or a fragment of a human, chimeric, or humanized antibody. Particularly suitable antibodies may include MAb 679, MAb 734, MAb Mu-9, and MAb MN-14. In addition, the binding molecule may include a fusion protein. Where the binding molecule includes a fragment of an antibody, the binding molecule may include one or more CDRs of a selected antibody. For example, the binding molecule may include the CDRs of MAb 679, MAb 734, MAb Mu-9, or MAb MN-14.

The binding molecule may specifically bind a variety of antigens. However, particular suitable antigens include carcinoembryonic antigen, tenascin, epidermal growth factor receptor, platelet derived growth factor receptor, fibroblast growth factor receptors, vascular endothelial growth factor receptors, gangliosides, HER/2neu receptors, and mixtures thereof. More specifically, the antigen may be selected from colon-specific antigen-p (CSAp), carcinoembryonic antigen (CEA), CD4, CD5, CD8, CD14, CD15, CD19, CD20, CD21, CD22, CD23, CD25, CD30, CD45, CD74, CD80, HLA-DR, Ia, Ii, MUC 1, MUC 2, MUC 3, MUC 4, NCA, EGFR, HER 2/neu, PAM-4, TAG-72, EGP-1, EGP-2, A3, KS-1, Le(y), S100, PSMA, PSA, tenascin, folate receptor, VEGF, PIGF, ILGF-1, necrosis antigens, IL-2, IL-6, T101, MAGE, and combinations thereof.

Immunomodulators or cytokines may be administered in addition to the primary therapeutic agent. For example, the immunomodulator or cytokine may include IL-1, IL-2, IL-3, IL-6, IL-10, IL-12, IL-18, IL-21, interferon-α, interferon-β, interferon-γ, G-CSF, and GM-CSF, or mixtures thereof.

It may be desirable to administer an anti-angiogenic agent in addition to the primary therapeutic agent. The anti-angiogenic agent may be selected from angiostatin, endostatin, baculostatin, canstatin, maspin, anti-VEGF antibodies, anti-placental growth factor antibodies, anti-vascular growth factor antibodies, and mixtures thereof.

In another embodiment, a therapeutic or diagnostic metal is administered in addition to the primary therapeutic agent. Suitable metals may include zinc, aluminum, gallium, lutetium, palladium, boron, gandolinium, uranium, manganese, iron, chrominum, copper, cobalt, nickel, dysprosium, rhenium, europium, terbium, holmium, neodymium, and combinations thereof. It may also be desirable to administer a paramagnetic ion in addition to the primary therapeutic agent (e.g., chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), or combinations thereof).

Desirable therapeutic and/or diagnostic agents may also include one or more agents for photodynamic therapy. For example, the agent may be a photosensitizer, such as molecule that includes a benzoporphyrin monoacid ring A (BDP-MA), tin etiopurpurin (SnET2), sulfonated aluminum phthalocyanine (AlSPc), orlutetium texaphyrin (Lutex).

In addition to administering the primary therapeutic agent, it may be desirable to administer one or more diagnostic agents for performing magnetic resonance imaging (MRI), for example, an image enhancing agent. Suitable image enhancing agents may include gadolinium ions, lanthanum ions, manganese ions, iron, chromium, copper, cobalt, nickel, fluorine, dysprosium, rhenium, europium, terbium, holmium, neodymium, or mixtures thereof.

It may also be desirable to administer one or more radioopaque materials or contrast agents for X-ray or computed tomography (CT). Suitable radioopaque materials or contrast agents include barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof.

In a further embodiment, it may be desirable to administer one or more contrast agents for performing an ultrasound imaging procedure. The contrast agent may include a liposome or dextran, and the liposome may be gas-filled.

In addition to administering the primary therapeutic agent, other therapeutic and/or diagnostic methods may be further performed such as an operative, intravascular, laparoscopic, or endoscopic procedure.

Also disclosed are kits that include the conjugates and/or complexes together with a pharmaceutically acceptable excipient to form a therapeutic agent. The kit may include an implement for administering the therapeutic agent. In addition, the kit may include one or more supplemental therapeutic agents and/or diagnostic agents.

More specifically, the present invention provides a conjugate comprising one or more moieties having ribonucleolytic activity and one or more folate receptor ligands. The invention further provides a complex comprising one or more moieties having ribonucleolytic activity conjugated to one or more histidine tags, one or more folate receptor ligand conjugated to one or more nitrolotriacetic acid residues, and nickel cations. The one or more moieties may contain one or more recombinant RNase molecules. The one or more moieties may comprise onconase and/or rapLR1. The one or more moieties preferably have pyroglutamate as an N-terminal residue. The one or more folate receptor ligands may be, for example, folic acid, methotrexate, or a folate analog that binds to the folate receptor. The one or more folate receptor ligands may be conjugated to the one or more moieties by a linker that comprises diisocyanate, diisothiocynate, carbodiimide, bis(hydroxysuccinimide) ester, maleimide-hydroxysuccinimide ester, glutaraldehyde, or a combination thereof. The one or more folate receptor ligands may be conjugated to the one or more moieties at one or more lysine, histidine, or cysteine residues.

These complexes may further comprise a peptide that includes one or more molecules selected from antigenic molecules, haptens, hard acid chelators, and soft acid chelators, wherein the one or more folate receptor ligands and the one or more nitrolotriacetic acid residues are conjugated to the peptide. The complex may further comprise one or more binding molecules, wherein the one or more binding molecules include one or more arms that specifically bind a targeted tissue and one or more arms that specifically bind the peptide. The invention also provides pharmaceutical compositions comprising a conjugate as described above and a pharmaceutically acceptable excipient.

The invention further provides a method of treating a disease, illness, or condition comprising administering a pharmaceutical composition as described above as a primary therapeutic agent to a subject in need thereof. The method may further comprise administering an additional therapeutic agent or a diagnostic agent before, concurrently, or after administering the primary therapeutic agent. The additional therapeutic agent or diagnostic agent may comprise a binding molecule, a drug, a prodrug, a toxin, an enzyme, an enzyme-inhibitor, a nuclease, a hormone, a hormone antagonist, an immunomodulator, a cytokine, an oligonucleotide, a chelator, a boron compound, a photoactive agent, a radionuclide, an anti-angiogenic agent, a dye, a radioopaque material, a contrast agent, a fluorescent compound, an enhancing agent, or combinations thereof. The oligonucleotide may comprise, for example, an anti-sense oligonucleotide, such as an anti-sense oligonucleotide against anti-bcl-2, or an interference RNA.

In these embodiments, the binding molecule may be multivalent and/or multispecific, for example bi-specific. The binding molecule may be an antibody. The additional therapeutic agent or diagnostic agent may comprise an antibody conjugated to a drug or a toxin. The additional therapeutic agent or diagnostic agent may comprise a radiolabelled antibody. The antibody or fragment thereof may comprises a human, chimeric, or humanized antibody or a fragment of a human, chimeric, or humanized antibody. The antibody may comprise MAb 679, MAb 734, MAb Mu-9, MN-14, or combinations thereof, or may comprise one or more CDRs selected from MAb 679, MAb 734, MAb Mu-9, and MN-14. The binding molecule may comprise a fusion protein.

In the therapeutic methods described above the disease, illness, or condition may be a malignant disease, a cardiovascular disease, an infectious disease, an inflammatory disease an autoimmune disease, a metabolic disease, or a neurological disease. The binding molecule may specifically binds a targeted tissue in, for example, a malignant disease. The targeted tissue may comprise an antigen selected from the group consisting of carcinoembryonic antigen, tenascin, epidermal growth factor receptor, platelet derived growth factor receptor, fibroblast growth factor receptors, vascular endothelial growth factor receptors, gangliosides, HER/2neu receptors and mixtures thereof. The targeted tissue may comprise a tumor, for example a tumor that produces or is associated with antigens selected from the group consisting of colon-specific antigen-p (CSAp), carcinoembryonic antigen (CEA), CD4, CD5, CD8, CD 14, CD15, CD19, CD20, CD21, CD22, CD23, CD25, CD30, CD45, CD74, CD80, HLA-DR, Ia, Ii, MUC 1, MUC 2, MUC 3, MUC 4, NCA, EGFR, HER 2/neu, PAM-4, TAG-72, EGP-1, EGP-2, A3, KS-1, Le(y), S100, PSMA, PSA, tenascin, folate receptor, VEGF, PIGF, ILGF-1, necrosis antigens, IL-2, IL-6, T101, MAGE, and combinations thereof. The targeted tissue may comprise a multiple myeloma, a B-cell malignancy, a T-cell malignancy, or combinations thereof. The B-cell malignancy may be, for example, an indolent form of B-cell lymphomas, an aggressive form of B-cell lymphoma, a chronic leukemia, multiple myeloma, or acute lymphatic leukemia. The targeted tissue may comprise a lymphoma including a non-Hodgkin's lymphoma or a Hodgkin's lymphoma, or the targeted tissue may comprise a solid tumor, such as a melanoma, a carcinoma, a sarcoma, a glioma, or combinations thereof. When the targeted tissue us a carcinoma it may be for example, esophageal, gastric, colonic, rectal, pancreatic, lung, breast, ovarian, urinary bladder, endometrial, cervical, testicular, renal, adrenal, or liver cancer, or a combination thereof.

The disease, illness, or condition may comprise a cardiovascular disease and the binding molecule may be specific for granulocytes, lymphocytes, monocytes, fibrin, or a mixture thereof. The cardiovascular disease may comprise myocardial infarction, ischemic heart disease, artheroschlerotic plaques, fibrin clots, emboli, or a combination thereof. The infectious disease may be a bacterial disease, fungal disease, parasitic disease, viral disease, protozoan disease, mycoplasmal disease, or combinations thereof. The infectious disease may caused, for example, by a pathogen selected from the group consisting of Microsporum, Trichophyton, Epidermophyton, Sporothrix schenckii, Cryptococcus neoformans, Coccidioides immitis, Histoplasma capsulatum, Blastomyces dermatitidis, Candida albicans, human immunodeficiency virus (HIV), herpes virus, cytomegalovirus, rabies virus, influenza virus, hepatitis B virus, Sendai virus, feline leukemia virus, Reovirus, polio virus, human serum parvo-like virus, simian virus 40, respiratory syncytial virus, mouse mammary tumor virus, Varicella-Zoster virus, Dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Anthrax bacillus, Streptococcus agalactiae, Legionella pneumophilia, Streptococcus pyogenes, Escherichia coli, Neisseria gonorrhoeae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis, Tetanus, a helminth, a malaria parasite, Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Onchocerca volvulus, Theileria parva, Taenia hydatigena, Taenia ovis, Taenia saginata, Echinococcus granulosus, Mesocestoides corti, Mycoplasma arthritidis, Mycoplasma hyorhinis, Mycoplasma orale, Mycoplasma arginini, Acholeplasma laidlawii, Mycoplasma salivarum, Mycoplasma pneumoniae, and combinations thereof.

The disease may be an autoimmune disease selected from the group consisting of acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosurn, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, perniciousanemia, rapidly progressive glomerulonephritis, psoriasis, fibrosing alveolitis, and combinations thereof. The disease, illness, or condition may be a metabolic disease or a neurological disease and the binding molecule may specifically bind, for example, an amyloid deposit.

In these embodiments, the drug, prodrug, or toxin may comprise aplidin, azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, semustine streptozocin, tamoxifen, taxanes, taxol, testosterone propionate, thalidomide, thioguanine, thiotepa, teniposide, topotecan, uracil mustard, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, or Pseudomonas endotoxin, or combinations thereof.

In those embodiments employing a radionuclide, the radionuclide may comprise ¹⁸F, ³²P, ³³P, ⁴⁵ Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Se, ⁷⁷As, ⁸⁶Y, ⁸⁹Sr, ⁸⁹Zr, ⁹⁰Y, ⁹⁴Tc, ^{94m}Tc, ⁹⁹Mo, ^{99m}Tc, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹PM, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb ²¹²Bi, ²¹²Pb, ²¹³Bi, ²²³Ra, ²²⁵Ac, or mixtures thereof. The radionuclide advantageously emits 70 to 700 keV gamma particles or positrons, or 25-4000 keV gamma particles and/or positrons. The radionuclide may be used for performing positron-emission tomography (PET).

In those embodiments employing an enzyme, the enzyme may be selected from carboxylesterases, glucuronidases, carboxypeptidases, beta-lactamases, phosphatases, nucleases, proteases, lipases, and mixtures thereof. In those embodiments employing an immunomodulat or or cytokine, the immunomodulat or or cytokine may comprise IL-1, IL-2, IL-3, IL-6, Il-10, IL-12, IL-18, IL-21, interferon-α, interferon-β, interferon-γ, G-CSF, and GM-CSF, or mixtures thereof.

In those embodiments employing an anti-angiogenic agent, it may be selected from the group consisting of angiostatin, endostatin, baculostatin, canstatin, maspin, anti-VEGF antibodies, anti-placental growth factor antibodies, anti-vascular growth factor antibodies, and mixtures thereof.

The methods described above optionally further comprise administering a diagnostic agent before, concurrently, or after administering the primary therapeutic agent. The diagnostic agent may comprise one or more agents for photodynamic therapy, such as a photosensitizer. The photosensitizer may comprise a benzoporphyrin monoacid ring A (BDP-MA), tin etiopurpurin (SnET2), sulfonated aluminum phthalocyanine (AlSPc) andor lutetium texaphyrin (Lutex). Thediagnostic agent may comprise one or more image enhancing agents and the method may further comprise performing magnetic resonance imaging (MRI). The image enhancing agent may comprise gadolinium ions, lanthanum ions, manganese ions, iron, chromium, copper, cobalt, nickel, fluorine, dysprosium, rhenium, europium, terbium, holmium, or neodymium, or mixtures thereof. The diagnostic agent may comprise one or more radiopaque materials or contrast agents for X-ray or computed tomography (CT). The radiopaque materials or contrast agents may include barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof. The diagnostic agent may comprise one or more contrast agents for performing an ultrasound imaging procedure, such as a liposome or dextran. The liposome may be gas-filled.

The methods of treatment may further comprise administering a metal selected from zinc, aluminum, gallium, lutetium, palladium, boron, gandolinium, uranium, manganese, iron, chrominum, copper, cobalt, nickel, dysprosium, rhenium, europium, terbium, holmium, neodymium, and combinations thereof. The methods of treatment may comprise administering a paramagnetic ion selected from chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), or combinations thereof.

The methods of treatment may further comprise performing an operative, intravascular, laparoscopic, or endoscopic procedure.

The invention further provides a method of treating and/or diagnosing a disease or condition that may lead to a disease in a patient comprising: administering to the patient a binding molecule, where the binding molecule has at least one arm that binds a targeted tissue and at least one other arm that binds a targetable construct; optionally, administering to the patient a clearing composition and allowing the composition to clear non-localized binding molecules from circulation; and administering to the patient a targetable construct comprising a pharmaceutical composition as described above.

The invention further provides a kit comprising a conjugate as described above and a pharmaceutically acceptable excipient together as a therapeutic agent. The kit may further comprise an implement for administering the therapeutic agent and/or one or more additional therapeutic agents. The kit may comprise one or more diagnostic agents.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows the nucleic acid sequence and amino acid sequence of NfM-onconase.

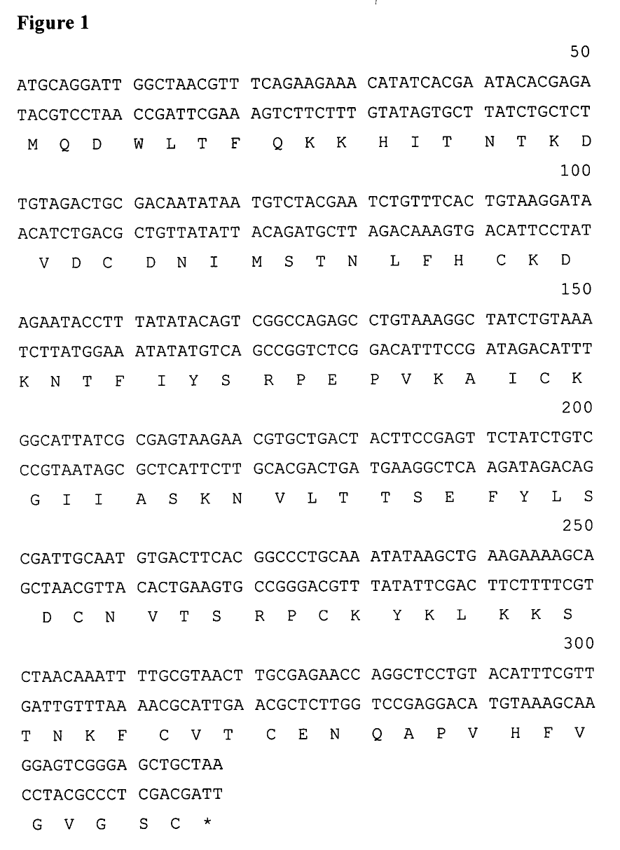

### DETAILED DESCRIPTION

Definitions

Unless otherwise defined, all technical and scientific terms used have the same meaning as commonly understood by one of ordinary skill in the art. In addition, the contents of all references cited herein are incorporated by reference in their entirety. For purposes of the present invention, the following terms are defined as follows:

Amino acids are referred to by name or by either their commonly known three-letter symbols or by the one-letter IUPAC symbols. Nucleotides are referred to by their commonly accepted single-letter codes.

“Conservatively modified variations” of a particular nucleic acid sequence refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Each codon in a nucleic acid except AUG which encodes methionine can be modified to yield a functionally identical molecule. The nucleic acid sequences described herein also encompass these alterations. A “variant” may include one or more “conservatively modified variations.”

“Conservatively modified variations” of an amino acid sequence include individual substitutions which alter a single amino acid or a small percentage of amino acids in an encoded sequence, where the alterations result in the substitution of an amino acid with a chemically similar amino acid. Conservative substitutions are well known to those of skill in the art. The following six groups each contain amino acids that are conservative substitutions for one another:
1. Alanine, Serine, Threonine
2. Aspartic acid, Glutamic Acid
3. Asparagine, Glutamine
4. Arginine, Lysine
5. Isoleucine, Leucine, Methionine, Valine, and
6. Phenylalanine, Tyrosine, Tryptophan.

“Conservatively modified variations” of an amino acid sequence also include deletions or additions of a single amino acid or a small percentage of amino acids in an encoded sequence, where the additions and deletions result in the substitution of an amino acid with a chemically similar amino acid. The amino acid sequences described herein also encompass these variations. A “variant” may include one or more “conservatively modified variations.”

The terms “isolated” or “biologically pure” refer to material which is substantially or essentially free from components which normally accompany it as found in its naturally occurring environment. The isolated material optionally comprises material not found with the material in its natural environment.

The term “nucleic acid” refers to a deoxyribonuclease or ribonucleotide polymer in either single- or double-stranded form and, unless otherwise limited, encompasses known analogues of natural nucleotides that hybridize to nucleic acids in a manner similar to naturally occurring nucleotides. Unless otherwise indicated, a particular nucleic acid sequence includes its complementary sequence.

An “expression vector” includes a recombinant expression cassette which includes a nucleic acid which encodes a polypeptide according to the invention which can be transcribed and translated by a cell. A recombinant expression cassette is a nucleic acid construct, generated recombinantly or synthetically, with a series of specified nucleic acid elements which permit transcription of a particular nucleic acid in a target cell. The expression vector can be part of a plasmid, virus, or nucleic acid fragment. Typically, the recombinant expression cassette portion of the expression vector includes a nucleic acid to be transcribed and a promoter operably linked thereto.

The term “recombinant” when used with reference to a protein indicates that a cell expresses a peptide or protein encoded by a nucleic acid whose origin is exogenous to the cell. Recombinant cells can express genes that are not found within the native (non-recombinant) form of the cell. Recombinant cells also can express genes found in the native form of the cell wherein the genes are re-introduced into the cell by artificial means, for example, under the control of a heterologous promoter.

The term “substantial identity” or “substantial similarity” in the context of a polypeptide indicates that a polypeptide comprises a sequence with at least 80%, more preferably 90%, and most preferably at least 95% identity with a reference sequence. Two polypeptides that are substantially identical means the one of the polypeptides is immunologically reactive with antibodies raised against the second peptide. Two nucleic acids are substantially identical is the two molecules hybridize to each other under stringent conditions. Generally, stringent conditions are selected to be about 5° C. to 20° C. lower than the thermal melting point (Tₘ) for a specific sequence at a defined ionic strength and pH. The Tₘ is the temperature (under defined ionic strength and pH) at which 50% of the target sequence hybridizes to a perfectly matched probe. However, nucleic acids which do not hybridize to each other under stringent conditions are still substantially identical if the polypeptides they encode are substantially identical.

A “targeting molecule” is a binding molecule, an antibody, cytokine or growth factor, an oligonucleotide (e.g., an antisense oligonucleotide or interference RNA), or ligand that is specific to a marker on a given cell type. Examples of antisense oligonucleotides and interference RNAs are disclosed in Kalota et al., Cancer Biol. Ther. 2004 January; 3(1); Tong et al., Clin. Lung Cancer 2001 February; 2(3):220-6; Dean et al., Oncogene 2003 Dec. 8; 22(56): 9087-96; Nahta et al., Semin. Oncol. 2003 October; 30(5 Suppl 16): 143-9; Patry et al., Cancer Res. 2003 Nov. 15; 63(22): 7679-88; Duxbury et al., Biochem Biophys Res Commun. 2003 Nov. 21; 311(3) 786-92; Crnkovic-Mertens et al., Oncogene 2003 Nov. 13; 22(51): 8330-6; Lipscomb et al., Clin Exp Metastasis 2003; 20(6): 569-76; Wall et al., Lancet 2003 Oct. 25; 362(9393): 1401-3; Bedford et al., Semin Cancer Biol 2003 August; 13(40): 301-8; Damm-Welk et al., Semin Cancer Biol. 2003 August; 13(4): 83-92; Duursma et al., Semin Cancer Biol. 2003 August; 13(4): 267-73, all of which are ncorporated herein by reference in their entireties. A targeting antigen can be used to specifically deliver an attached molecule to a given cell type, by preferentially associating with the marker associated with that cell type.

A “fusion protein” is a chimeric molecule formed by joining two or more polypeptides, for example, onconase and a targeting antigen or antibody. Onconase and the targeting antigen are typically joined through a peptide bond formed between the amino terminus of the targeting antigen and the carboxyl terminus of onconase, and are expressed recombinantly by a nucleic acid sequence encoding the fusion protein. A single chain fusion protein is a fusion protein that has a single contiguous polypeptide backbone.

A “chemical conjugate” is a conjugate formed by the chemical coupling of two molecules (e.g., onconase and a targeting antigen or antibody).

“A pharmaceutically acceptable carrier” is a material that can be used as a vehicle for administering a therapeutic or diagnostic agent, (e.g., onconase or a fusion protein), because the material is inert or otherwise medically acceptable, as well as compatible with the agent.

A binding molecule, as described herein, is any molecule that can specifically bind to an antigen. A binding molecule may include an antibody or a fragment thereof, such as F(ab′)₂, F(ab)₂, Fab′, Fab, Fv and the like, including hybrid fragments. Also useful are any subfragments that retain the hypervariable, antigen-binding region of an immunoglobulin. A binding molecule may also include an oligonucleotide (e.g., an antisense oligonucleotide or an interference RNA).

An antibody, as described herein, refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment.

An antibody fragment is a portion of an antibody such as F(ab)₂, F(ab)₂, Fab, Fab, Fv, sFv and the like. Regardless of structure, an antibody fragment binds with the same antigen that is recognized by the intact antibody. The term “antibody fragment” also includes any synthetic or genetically engineered protein that acts like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

A chimeric antibody is a recombinant protein that contains the variable domains including the complementarity determining regions (CDRs) of an antibody derived from one species, preferably a rodent antibody, while the constant domains of the antibody molecule is derived from those of a human antibody. For veterinary applications, the constant domains of the chimeric antibody may be derived from that of other species, such as a cat or dog.

A humanized antibody is a recombinant protein in which the CDRs from an antibody from one species; e.g., a rodent antibody, is transferred from the heavy and light variable chains of the rodent antibody into human heavy and light variable domains. The constant domains of the antibody molecule is derived from those of a human antibody.

A human antibody is an antibody of totally human composition obtained from various sources, including transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. (See, e.g., McCafferty et al., Nature 348:552-553 (1990) for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275, which are incorporated in their entirety by reference).

An effector is an atom, molecule, or compound that brings about a chosen result. An effector may include a therapeutic agent and/or a diagnostic agent as described herein.

A therapeutic agent is an atom, molecule, or compound that is useful in the treatment of a disease. Non-limiting examples of therapeutic agents include binding molecules (e.g., antibodies or antibody fragments), drugs, toxins, enzymes, nucleases, hormones, immunomodulators, oligonucleotides (e.g., antisense oligonucleotide or interference RNA), chelators, boron compounds, photoactive agents or dyes and radioisotopes.

A diagnostic agent is an atom, molecule, or compound that is useful in diagnosing a disease. Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). U.S. Pat. No. 6,331,175 describes MRI technique and the preparation of antibodies conjugated to a MRI enhancing agent and is incorporated in its entirety by reference. Preferably, the diagnostic agents are selected from the group consisting of radioisotopes, enhancing agents for use in magnetic resonance imaging, and fluorescent compounds. In order to load an antibody component with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, e.g., ethylenediaminetetraacetic acid (EDTA), diethylenetriaminepentaacetic acid (DTPA), porphyrins, polyamines, crown ethers, bis-thiosemicarbazones, polyoximes, and like groups known to be useful for this purpose. Chelates are coupled to the peptide antigens using standard chemistries. The chelate is normally linked to the antibody by a group which enables formation of a bond to the molecule with minimal loss of immunoreactivity and minimal aggregation and/or internal cross-linking. Other, more unusual, methods and reagents for conjugating chelates to antibodies are disclosed in U.S. Pat. No. 4,824,659 to Hawthorne, entitled “Antibody Conjugates”, issued Apr. 25, 1989, the disclosure of which is incorporated herein in its entirety by reference. Particularly useful metal-chelate combinations include 2-benzyl-DTPA and its monomethyl and cyclohexyl analogs, used with diagnostic isotopes in the general energy range of 60 to 4,000 keV. Some useful diagnostic nuclides may include, such as ¹⁸F, ⁵²Fe, ⁶²Cu, ⁶⁴Cu, ⁶⁷Cu, ⁶⁷Ga, ⁶⁸Ga, ⁸⁶Y ⁸⁹Zr, ⁹⁴Tc, ⁹⁴ mTc, ^{99m}Tc, or ¹¹¹In, ¹²⁴I, and ¹³¹I. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals, most particularly with radionuclides of gallium, yttrium and copper, respectively. Such metal-chelate complexes can be made very stable by tailoring the ring size to the metal of interest. Other ring-type chelates such as macrocyclic polyethers, which are of interest for stably binding nuclides, such as ²²³Ra for radioimmunotherapy (“RAIT”) may be used.

An immunoconjugate is a conjugate of a binding molecule (e.g., an antibody component) with an atom, molecule, or a higher-ordered structure (e.g., with a carrier, a therapeutic agent, or a diagnostic agent). The diagnostic agent can comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A naked antibody is an antibody that is not conjugated to any other non-antibody agent.

A carrier is an atom, molecule, or higher-ordered structure that is capable of associating with a therapeutic or diagnostic agent to facilitate delivery of the agent to a targeted cell. Carriers may include molecules such as lipids or polymers (e.g., amphiphilic lipids that are capable of forming higher-ordered structures, or carbohydrates such as dextran), or higher-ordered structures themselves, such as micelles, liposomes, or nanoparticles.

As used herein, the term antibody fusion protein is a recombinantly produced antigen-binding molecule in which two or more of the same or different single-chain antibody or antibody fragment segments with the same or different specificities are linked. Valency of the fusion protein indicates how many binding arms or sites the fusion protein has to a single antigen or epitope; i.e., monovalent, bivalent, trivalent or multivalent. The multivalency of the antibody fusion protein means that it can take advantage of multiple interactions in binding to an antigen, thus increasing the avidity of binding to the antigen. Specificity indicates how many antigens or epitopes an antibody fusion protein is able to bind; i.e., monospecific, bispecific, trispecific, multispecific. Using these definitions, a natural antibody, e.g., an IgG, is bivalent because it has two binding arms but is monospecific because it binds to one epitope. Monospecific, multivalent fusion proteins have more than one binding site for an epitope but only binds with one epitope, for example a diabody with two binding site reactive with the same antigen. The fusion protein may comprise a single antibody component, a multivalent or multispecific combination of different antibody components or multiple copies of the same antibody component. The fusion protein may additionally comprise an antibody or an antibody fragment and a therapeutic agent. Examples of therapeutic agents suitable for such fusion proteins include immunomodulators (“antibody-immunomodulator fusion protein”) and toxins (“antibody-toxin fusion protein”). One preferred toxin comprises a ribonuclease (RNase), preferably a recombinant RNase such as onconase or rapLR1.

A multispecific antibody is an antibody that can bind simultaneously to at least two targets that are of different structure, e.g., two different antigens, two different epitopes on the same antigen, or a hapten and/or an antigen or epitope. One specificity would be for a B-cell, T-cell, myeloid-, plasma-, and mast-cell antigen or epitope. Another specificity could be to a different antigen on the same cell type, such as CD20, CD19, CD21, CD23, CD46, CD80, HLA-DR, CD74, and CD22 on B-cells. A multivalent antibody is an antibody that can bind simultaneously to at least two targets that are of the same or different structure. Multispecific, multivalent antibodies are constructs that have more than one binding site of different specificity. For example, a diabody, where one binding site reacts with one antigen and the other with another antigen.

A bispecific antibody is an antibody that can bind simultaneously to two targets which are of different structure. Bispecific antibodies (bsAb) and bispecific antibody fragments (bsFab) have at least one arm that specifically binds to, for example, a B-cell, T-cell, myeloid-, plasma-, and mast-cell antigen or epitope and at least one other arm that specifically binds to a targetable conjugate that bears a therapeutic or diagnostic agent. A variety of bispecific fusion proteins can be produced using molecular engineering. In one form, the bispecific fusion protein is monovalent, consisting of, for example, a scFv with a single binding site for one antigen and a Fab fragment with a single binding site for a second antigen. In another form, the bispecific fusion protein is divalent, consisting of, for example, an IgG with a binding site for one antigen and two scFv with two binding sites for a second antigen.

Preparation of Conjugates

Preparation of Recombinant Onconase-Encoding Genes

The disclosed conjugates and complexes may be prepared by conventional methods. For example, a nucleic acid that encodes native onconase may be prepared by cloning and restriction of appropriate sequences, or using DNA amplification with polymerase chain reaction (PCR). Recombinant onconase molecules and the preparation of onconase-conjugates have been previously disclosed. See U.S. application Ser. No. 10/117,342, filed Apr. 8, 2002; U.S. Pat. No. 6,399,086; U.S. Pat. No. 6,083,677; U.S. Appl. Ser. No. 60/028,430, filed Oct. 17, 1996; U.S. Pat. No. 6,653,104; U.S. Pat. No. 6,395,276; U.S. Appl. Ser. No. 60/046,895, filed May 2, 1997; U.S. application Ser. No. 10/153,882, filed May 24, 2002; U.S. application Ser. No. 09/265,901, filed Mar. 11, 1999; and U.S. Appl. Ser. No. 60/077,577 filed Mar. 11, 1998, which are incorporated herein by reference in their entireties.

The amino acid sequence of onconase can be obtained from Ardelt et al., J. Biol. Chem., 256: 245 (1991), and cDNA sequences encoding native onconase, or a conservatively modified variation thereof, can be gene-synthesized by methods similar to the en bloc V-gene assembly in hLL2 humanization. Leung et al., Mol. Immunol., 32: 1413 (1995). For expression in E. coli, a translation initiation codon ATG, is placed in-frame preceding the onconase cDNA sequence. The translated protein then contains an additional Met at the −1 position. A histidine tag can be added to the COOH-terminus of a molecule by adding, in frame, preferably at least six codons for histidine, (i.e., CAU and CAC).

Alternatively, nucleic acid that encodes native onconase may be synthesized in vitro. Chemical synthesis produces a single-stranded oligonucleotide. This may be converted to a double-stranded DNA by hybridization with a complementary sequence, or by polymerization with a DNA polymerase using the single strand as a template. While chemical synthesis is limited to sequences of about 100 bases, longer sequences may be obtained by ligating shorter sequences.

Expression of Recombinant Onconases

As noted, a gene encoding native onconase, or a conservatively modified variation thereof, may be modified to include a codon for N-formyl-methionine at the N-terminus. The thus-obtained NfM-onconase gene may be operably linked to a suitable E. coli promoter, such as the T7, trp, or lambda promoter, and inserted in an expression cassette. Preferably a ribosome binding site and transcription termination signal also are included in the expression cassette. An expression vector that contains the cassette is transferred into an E. coli expression host by methods known to those of skill in the art. Transformed cells can be selected resistance to antibiotics conferred by marker genes contained in the expression vector.

The transformed E. coli host expresses NfM-onconase, which may be contained in an inclusion body. Although onconase possesses potent RNase activity, NfM-onconase does not. This is because the N-terminal pyroglutamate on onconase is part of the active site, as demonstrated by the crystal structure of onconase. The inherent nature of the bacterial expression system, which requires an N-terminal Met, means that the bacterial expression product is inactive. This enables recombinant expression of NfM-onconase in bacterial expression systems. While NfM-onconase is not toxic, it also may be expressed as inactive inclusion bodies.

NfM-onconase can be isolated and purified according to standard procedures, including ammonium sulfate precipitation, affinity columns, column chromatography, and gel electrophoresis. Substantially pure compositions of at least about 90-95% homogeneity, and preferably 98-99% homogeneity, are preferred.

Following purification of the NfM-onconase, and refolding of the molecule if it was expressed in an inclusion body, the N-formyl-methionine may be removed by digestion with aminopeptidase. A suitable aminopeptidase is Aeromonas aminopeptidase, as disclosed in Shapiro et al. Anal. Biochem. 175:450-461 (1988). Incubation of the resulting product results in spontaneous cyclization of the N-terminal glutamine residue, to form a molecule having the structure and function of native onconase.

Preparation of Conjugates and Fusion Proteins

The recombinantly-produced onconase can be used as an alternative or complement to existing toxins, as well as for construction of effective chemical conjugates and fusion proteins of high potency and low immunogenicity. In this regard, chemical conjugates and fusion proteins may include a molecule having the structure and function of native onconase and a folate receptor ligand (e.g., folic acid, methotrexate, or a folate analog that binds to the folate receptor). The folate receptor ligand may function as a targeting (and/or internalizing molecule), which targets the onconase to a the folate receptor.

A chemical conjugate of recombinantly-produced onconase according to the invention and a folate receptor ligand can be produced by standard chemical conjugation procedures. See, e.g., Leamon, et al., (1993) J. Biol. Chem. 267, 24966-71; Reddy et al., Blood, 1999, 93, 3940-48; and Wang, Mathias, and Siegel supra. The chemical conjugate can be formed by covalently linking the folate receptor ligand to onconase or a derivate thereof, either directly or through a short or long linker molecule, through one or more functional groups on the folate receptor ligand, (e.g., amine, carboxyl, phenyl, thiol, and/or hydroxyl groups) to form a covalent conjugate. Various conventional linkers can be used, e.g., diisocyanates, diisothiocyanates, carbodiimides, bis(hydroxysuccinimide) esters, maleimide-hydroxysuccinimide esters, glutaraldehyde and the like.

Preparation and Use of Pharmaceutical Compositions

Recombinantly-produced onconase conjugates may be formulated into pharmaceutical compositions for treating tumors or killing other unwanted cell types in vivo. The compositions are particularly suitable for parenteral administration, such as intravenous administration. In this context, the compositions comprise a solution of the conjugate dissolved in a pharmaceutically acceptable carrier, preferably an aqueous carrier such as buffered saline. These solutions are sterile and may contain auxiliary substances such as pH adjusting and buffering agents and toxicity adjusting agents.

A preferable dosage of the conjugate is about 0.1 to 10 mg per patient per day, although dosages of up to 300 mg per patient per day may be used, particularly when the drug is administered locally, and not into the bloodstream. Like native onconase, the onconase conjugate is readily internalized in cells, has anti-tumor effects in vivo, and preferentially kills rapidly dividing tumor cells. Chemical conjugates provide for more specific targeting of the recombinantly-produced onconase to particular cells.

In therapeutic applications, compositions are administered to a patient suffering from a disease, in a cytotoxic amount, which is defined as an amount sufficient to kill cells of interest. An amount successful to accomplish this is defined as a “therapeutically effective amount.” The exact amount will depend on the severity of the disease and the general state of the patient's health. Single or multiple administrations of the compositions may be administered depending on the dosage required.

Onconase conjugates can also be used to treat populations of cells in vitro. For example, it may be used selectively to kill unwanted cell types in bone marrow prior to transplantation into a patient undergoing marrow ablation.

The conjugates and/or complexes described herein may be administered together with one or more binding molecules that may recognize a variety of antigens. Exemplary antigens are glycosylated cell surface antigens that are expressed on solid tumors, such as carcinoembryonic antigen (CEA). CEA represents an attractive antigenic target for several reasons. CEA is a tumor-associated antigen that it is absent or poorly expressed by normal tissues and highly expressed by the vast majority of carcinomas of breast, colon, lung, pancreas, ovarian, and medullary thyroid origin. High mortality rates coupled with suboptimal diagnostic and therapeutic options for these malignancies result in a serious, persistent public health problem. Chemical conjugates of onconase-folate administered with one or more antibodies to glycosylated surface antigens, particularly anti-CEA antibodies, is one such example.

CEA is a glycosylated cell surface protein of approximately 180 kDa, and is a solid tumor antigen that has been extensively studied clinically, both as a circulating tumor marker and as an antigenic target for radiolabeled mAbs for imaging and therapy. A number of anti-CEA antibodies have been under study in phase I-III clinical diagnostic and therapeutic trials. MN14 mAb is an exemplary anti-CEA mAb. A humanized version of this mAb, hMN-14, in which human constant and framework regions replace the corresponding mouse sequences, has been constructed and expressed and may be particularly suitable for administering with the conjugates described herein. Other useful antigens include CD74 and EGP-1, which may facilitate internalization of the bound antibody. Antibodies that recognize CD74 include LL 1, the use of which is described in U.S. Pat. No. 6,458,933; U.S. Pat. No. 6,395,276; U.S. Pat. No. 6,083,477; and U.S. 2003-0103982. Antibodies that recognize EGP-1 include RS7, which is described in U.S. Ser. No. 10/377,121; U.S. Pat. No. 5,635,603; and Stein et al., 1990, Cancer Res., 50, 1330-1336.

The binding molecule may include one or more V_{K} and V_{H} sequences of a particular antibody. For example, the V_{K} and V_{H} sequences of an antibody can be cloned using PCR-amplification. See, e.g., Orlandi et al., PNAS, 86: 3833 (1989). The binding molecule may also include fragments of antibodies such as F(ab′)₂, F(ab)₂, Fab′, Fab, Fv and the like, including hybrid fragments. Also useful are any subfragments that retain the hypervariable, antigen-binding region of an immunoglobulin, including genetically-engineered and/or recombinant proteins, whether single-chain or multiple-chain, which incorporate an antigen binding site and otherwise function in vivo as targeting molecules in substantially the same way as natural immunoglobulin fragments. Single-chain binding molecules are disclosed in U.S. Pat. No. 4,946,778. Fab′ antibody fragments may be conveniently made by reductive cleavage of F(ab′)₂ fragments, which themselves may be made by pepsin digestion of intact immunoglobulin. Fab antibody fragments may be made by papain digestion of intact immunoglobulin, under reducing conditions, or by cleavage of F(ab)₂ fragments which result from careful papain digestion of whole Ig. The fragments may also be produced by genetic engineering.

As noted above, the complexes may include one or more peptides that include one or more folate receptor ligands and one or more nitrilolotriacetic acid residues. Therapeutic and/or diagnostic peptides are described in U.S. application Ser. No. 10/150,654, filed May 17, 2002; Ser. No. 09/823,746, filed Apr. 3, 2001; Ser. No. 09/382,186, filed Aug. 23, 1999; and Ser. No. 09/337,756, filed Jun. 22, 1999, which are incorporated herein by reference in their entireties.

The present invention, thus generally described, will be understood more readily by reference to the following examples, which are provided by way of illustration and are not intended to be limiting of the present invention.

### EXAMPLES

### Example 1

### Synthesis of PCR-Amplified DNA Encoding Onconase Derivatives

A 139-mer DNA nucleotide, ONCO-N, with the sense strand sequence [5′-TGG CTA ACG TTT CAG AAG AAA CAT ATC ACG AAT ACA CGA GAT GTA GAC TGG GAC AAT ATA ATG TCT ACG AAT CTG TTT CAC TGT AAG GAT AAG AAT ACC TTT ATA TAC AGT CGG CCA GAG CCT GTA AAG GCT ATC TGT A-3′] encoding an N-terminal sequence (46 amino acids) of recombinant onconase is synthesized by an automated DNA synthesizer (Applied Biosystem 392 DNA/RNA Synthesizer) and used as the template for PCR-amplification with the flanking primers ONNBACK [5′-AAG CTT CAT ATG CAG GAT TGG CTA ACG TTT CAG AAG AAA-3′, and ONNFOR [5′-CTT ACT CGC GAT AAT GCC TTT ACA GAT AGC CTT TAC AGG CTC TG-3′]. The resultant double-stranded PCR product contains cDNA sequence that encodes for 54 amino acid residues of the N-terminal half of onconase. ONNBACK contains the restriction sites HindIII (AAAGCTT) and NdeI (CATATG) to facilitate subcloning into either a staging vector or for in-frame ligation (NdeI site) into the bacterial expression vector. The NruI site (TCGCGA) is incorporated in the ONNFOR primer to facilitate in-frame ligation with the cDNA encoding the C-terminal half of onconase.

Similarly, a 137-mer DNA nucleotide, ONCO-C, with the sense-strand sequence [TGC TGA CTA CTT CCG AGT TCT ATC TGT CCG ATT GCA ATG TGA CTT CAC GGC CCT GCA AAT ATA AGC TGA AGA AAA GCA CTA ACA AAT TTT GCG TAA CTT GCG AGA ACC AGG CTC CTG TAC ATT TCG TTG GAG TCG GG-3′] encoding the C-terminal sequence (46 amino acids) of onconase is synthesized and PCR-amplified by the primers ONCBACK[5′-ATT ATC GCG AGT AAG AAC GTG CTG ACT ACT TCC GAG TTC TAT-3′] and ONCFOR[5′-TTA GGA TCC TTA GCA GCT CCC GAC TCC AAC GAA ATG TAC-3′]. Modified C-terminal primers that include a six-histidine tag can also be used. The final double-stranded PCR product contained a cDNA sequence that encoded 51 amino acids of the rest of the C-terminal half of onconase. A NruI site allowed in-frame ligation with the N-terminal half of the PCR-amplified DNA incorporated in ONCBACK. A stop codon (shown in bold italics) and BamHI restriction sites (underlined) for subcloning into staging or expression vectors were included in the ONCFOR sequence.

The PCR-amplified DNA encoding the N- and C-terminal half of NfM-onconase, after being treated with the appropriate restriction enzymes, were joined at the NruI sites and subcloned into a staging vector, e.g., pBluescript from Stratagene. The ligated sequence should encode a polypeptide of 105 amino acids with an N-terminal Met.

### Example 2

### Expression and Purification of NfM-Onconase

NfM-onconase cDNA is digested and cloned into an expression vector such as pET (Novagen, Madison, Wis.), which has a T7 promoter. The final NfM-onconase expression vector is verified by sequencing and designated rOncopET.

Large scale expression of the recombinant protein from the T7-driven rOncopET vector requires an appropriate host E. coli, such as BL21, which contains a DE3 lysogen, as described above. rOncopET vector is used to transform competent BL21/DE3 cells by electroporation. Colonies that survive selection on Amp agar plates are picked and grown in a shaker incubator at 37° C. in 3 ml of LB-Amp. After incubation for 8-10 hours, 100 μl of the culture is transferred to 25 ml of superbroth (LB supplemented with 0.5% glucose, 1.6 mM MgSO₄ and 100 μg/ml ampicillin) in a 500 ml E-flask to increase aeration while shaking. The culture is incubated overnight in the shaker incubator at 37° C. The culture then is transferred into one liter of superbroth and further incubated in a shaker incubator at 37° C. IPTG at a final concentration of 1 mM is added into the culture when the OD₆₅₀ of the culture reaches 1 (approx. 2.5 hours). Induction is allowed to proceed for 1-3 hours before the culture is terminated for inclusion body isolation. Ten ill of the culture is analyzed under reducing conditions in 15% SDS-PAGE gel. Colonies with the highest level of induction are kept as stock culture and stored frozen at −70° C.

Inclusion body isolation entails lysis of cells by homogenization in the presence of lysozymes to release the incudions bodies as insoluble pellets. The washed inclusion bodies are dissolved in denaturing buffer that contains 7 M guanidine-HCl. Disulfide bonds are reduced by dithiothreitol and then are refolded by dropwise dilution of the denatured protein in renaturing buffer that contains arginine-HCL and oxidized glutathione.

### Example 3

### Expression and Purification of NfM-Onconase

A 6-liter culture equivalent of renatured inclusion bodies from Example 2 is purified. Harvested cell paste is resuspended using a Tisuemizer tip (Thomas, Swedesboro, N.J.) in TES buffer (50 mM Tris, pH 8, 100 mM NaCl, and 20 mM EDTA) containing 180 μg/ml lysozyme. After incubating at 22° C. for one hour, the cells are resuspended again and centrifuged at 27,000 g for 50 minutes. The pellet is washed by resuspension and centrifugation three or four times with TES buffer containing 2.5% Triton-X-100 and then four times with TES. The inclusion bodies are resuspended in 5 to 10 ml of denaturation buffer (7 M guanidine:HCl, 0.1 M Tris, pH 8.0, and 5 mM EDTA) by sonication or tissuemizing and diluted to a protein concentration of 10 mg/ml.

The protein is reduced with dithiothreitol (65 mM) for 4 to 24 hours at 22° C. and rapidly diluted in a thin stream into refolding buffer (0.1 M Tris, pH 8.0, 0.5 arginine:HCl, 2 mM EDTA, and 0.9 mM oxidized glutathione). After incubating at 10° C. for 36 to 72 hours, the refolded NfM-onconase is dialyzed against 0.15 M sodium acetate (pH 5), and is loaded onto a HiLoad 16/20 SP cation exchange FPLC column. The buffer is then exchanged into 0.15 M sodium acetate (pH 5), and the precipitates are removed by centrifugation. Elution with a 0-1 M linear gradient of sodium chloride is used, and fractions corresponding to absorbance peaks are analyzed by SDS-PAGE (15%). The eluted products are dividing into fractions for further analysis.

### Example 4

### Removal of Met from NfM-Onconase and NfM-Onconase Fusion Proteins

The N-terminal Met residues of NfM-onconase, NfM-onconase-hMN14-scFv and NfM-onconase-hLL2-scFv are removed according to Shapiro et al. (1988). 100-200 μg/ml of purified and renatured proteins are incubated with 0.5 μg/ml Aeromonas proteolytica aminopeptidase (Sigma Chemicals, St. Louis, Mo.) in 200 mM sodium phosphate, pH 7.5 for 18 hours at 37° C.

### Example 5

### Preparation of Onconase-Folate Conjugates

Onconase-folate conjugates may be prepared as described by Leamon, et al., (1993) J. Biol. Chem. 267, 24966-71. Folate is dissolved in anhydrous dimethyl sulfoxide and activated with w 5-fold molar excess of 1-ethyl-3-(3-dimethylamineopropyl) carbodiimide for 1 h at 23° C. Onconase is dissolved in 0.1 M Na₂HPO₄, 0.1 M boric acid, pH 8.5. A 5-8-fold molar excess of the activated folate is then added to the onconase solution, and the conjugation reaction is allowed to proceed at 23° C. for 30 min. Unreacted material is separated from the conjugate using a Sephadex G-25 column equilibrated in phosphate-buffered saline, pH 7.4 (PBS). Samples are filtered through 0.2 μm syringe filters and then assayed for protein content using standard methods such as a bicinchoninic acid assay (BCA) kit (Pierce Chemical Co.). Peak fractions are pooled and analyzed by SDS-PAGE to determine the amount of free ligand and recombinant onconase in the conjugates. The extent of folate conjugate may be determined as described by Leamon et al., (1991) Proc. Natl. Acad. Sci. U.S.A. 88, 5572-5576.

### Example 6

### Synthesis of Peptides that Include Nitrilotriacetic Acid Residues

The peptide was synthesized by solid phase peptide synthesis on Sieber Amide resin using the Fmoc methodology. The amino acids were coupled to the resin using six equivalents of amino acid relative to the resin loading. The activation method used diisopropylcarbodiimide (DIC) and HOBt reacting overnight. The amino acids added to the resin were (in order), Fmoc-Lys(Aloc)-OH, Fmoc-Lys(Aloc)-OH, Fmoc-Lys(Aloc)-OH, Fmoc-Cys(Trt)-OH and acetic anhydride. The side chain Aloc protecting groups were then removed with Pd[P(PH)₃]₄ and HSn(bu)₃. The side chains of the lysine were then reacted with Fmoc-Asp-OBut(Fmoc L-Aspartic Acid α-t-butyl ester. The Fmoc group was then removed from the Aspartic acids and the nitrogens were per-alkylated by the addition of an excess of t-butylbromoacetate in the presence of diisopropylethylamine (reacting overnight at room temperature). The peptide was then cleaved, deprotected, and purified by HPLC. The peptide was designated IMP 267.

### Example 7

### Conjugation of Folic Acid to the Synthesized Peptide (IMP 267)

Folic acid is dissolved in DMSO and activated with one equivalent of DIC to form an anhydride which is reacted with 3-[2-pyridyldithio]proprionyl hydrazide (PDPH). The reaction product is purified by reverse phase HPLC and the fractions containing the reaction product are lyophilized. The reaction product is then mixed with the thiol containing peptide (IMP 267) at pH 5 to 9 to form the disulfide-linked peptide folate conjugate.

### Example 8

### Affinity Analysis of Peptides that Include Nitrilotriacetic Acid Residues and His-tagged Onconase

IMP 267 was bound to a Biacore® C1 sensor chip first by dissolving 0.0040 g of IMP 267 in 690 μL of 0.01 M, pH 4.3 formate buffer. The chip was activated for coupling of the peptide via a disulfide linkage using pyridyldithioelthylamine (“PDEA”) as described by Biacore. The Biacore® C1 chip was activated with a 10 μL aliquot of the recommended 1-ethyl-3-(3-dimethylaminoprolyl) carbodiimide (“EDC”)/N-hydroxysuccinimide (“NHS”) mixture at 5 μL/min in flow cells 1 & 2. The recommended PDEA solution, 35 μL (0.0047 g PDEA in 0.1 M, pH 8.5 Borate buffer) was added to the chip at flow cells 1 & 2. The peptide, 35 μL was added to flow cells 1 & 2 and the chip was quenched with an aliquot of cysteine (made from 0.0026 g cys, 0.0268 g NaCl, and 433 μL 0.1 M, pH 4.3 formate buffer) into cells 1 & 2. The chip was activated by adding nickel to only one flow cell using the other flow cell, without nickel, as the control. The activation was done at 40 μL/min using 100 μL of a 500 μM NiCl₂ solution in NTA buffer. A kinetics experiment was performed using the following concentrations of onconase bearing a six His tag (50 nM, 25 nM, 10 nM, 5 nM, 1 nM and 0 nM). The affinity was found to be K_{d}=1.15×10⁻⁹ M with a Chi² fit of 1.63. The affinity of His-tagged onconase is accessed similarly.

### Example 9

### Testing In Vitro Activity of Recombinant Onconase, Onconase-Conjugates, and Onconase-Complexes

The ability of onconase-conjugates to inhibit protein synthesis in a rabbit reticulocyte lysate assay is assessed using protocols described by St. Clair et al., PNAS USA, 84: 8330 (1987). All fractions to be tested are first passed through Superose 12 FPLC, however, because aminopeptidase has a molecular weight of 29 kD and NfM-onconase has a molecular weight of 12 kD, the two cannot be separated by size exclusion chromatography using Superose 12 FPLC.

Native onconase (AlfaCell) and various fractions of recombinant onconase (+/−aminopeptidase treatment) and onconase-folate conjugates are compared in the rabbit reticulocyte lysate assay. After incubating the assay mixture with or without ribonucleases, ³²Met incorporation is measured in a scintillation counter to assess the rate of protein synthesis. Protein concentrations of different samples are determined at the same time by BCA assay (Pierce) using BSA as the standard.

The ability of the recombinant onconase and onconase-folate conjugates to inhibit protein synthesis in cell lines is also tested. Suitable cell lines include Daudi, Raji, CA-46, and the human T cell line, Hut 102. Cells are plated at concentrations of 2×10⁵ cells/ml in 96-well microtiter plates overnight in the appropriate complete media. The complete media is replaced by serum-free and leucine-free media containing increasing concentrations of recombinant onconase and onconase-folate conjugate, for 16 hours followed by a 1 hour pulse with 0.1 μCi of [¹⁴C]leucine. Cells are harvested onto glass fiber filters using a cell harvester (Skaron), washed with water, dried with ethanol, and counted. Cytotoxic/cytostatic effects on the cells are assessed by performing the experiments in parallel, except that the cells are exposed to a 1 hour pulse with 0.5 μCi of [³H]thymidine. IC₅₀ are calculated to estimate the activity of the onconase and/or conjugates.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the invention.

It will be readily apparent to one skilled in the art that varying substitutions and modifications may be made to the invention disclosed herein without departing from the scope and spirit of the invention. For example, a variety of different binding pairs can be utilized, as well as a variety of different therapeutic and diagnostic agents. Thus, such additional embodiments are within the scope of the present invention.

The invention illustratively described herein suitably may be practiced in the absence of any element or elements, limitation or limitations which is not specifically disclosed herein. Thus, for example, in each instance herein any of the terms “comprising”, “consisting essentially Of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

1. A conjugate comprising:
one or more moieties having ribonucleolytic activity; and
one or more folate receptor ligands.

2. The conjugate of claim 1, wherein the one or more moieties comprise a recombinant RNase molecule.

3. The conjugate of claim 1, wherein the one or more moieties have pyroglutamate as an N-terminal residue.

4. The conjugate of claim 1, wherein the one or more folate receptor ligands are folic acid, methotrexate, or a folate analog that binds to the folate receptor.

5. The conjugate of claim 1, wherein the one or more folate receptor ligands are conjugated to the one or more moieties by a linker that comprises diisocyanate, diisothiocynate, carbodiimide, bis(hydroxysuccinimide) ester, maleimide-hydroxysuccinimide ester, glutaraldehyde, or a combination thereof.

6. The conjugate of claim 1, wherein the one or more folate receptor ligands are conjugated to the one or more moieties at one or more lysine, histidine, or cysteine residues.

7. A composition comprising the conjugate of claim 1 and a pharmaceutically acceptable excipient.

8. A complex comprising:
one or more moieties having ribonucleolytic activity conjugated to one or more histidine tags;
one or more folate receptor ligand conjugated to one or more nitrilotriacetic acid residues;
and nickel cations.

9. The conjugate of claim 8, wherein the one or more moieties comprise a recombinant RNase molecule.

10. The conjugate of claim 1, wherein the one or more moieties have pyroglutamate as an N-terminal residue.

11. The complex of claim 8, further comprising a peptide that includes one or more molecules selected from antigenic molecules, haptens, hard acid chelators, and soft acid chelators, wherein the one or more folate receptor ligands and the one or more nitrilotriacetic acid residues are conjugated to the peptide.

12. The complex of claim 8, further comprising one or more binding molecules, wherein the one or more binding molecules include one or more arms that specifically bind a targeted tissue and one or more arms that specifically bind the peptide.

13. A composition comprising the complex of claim 8 and a pharmaceutically acceptable excipient.

14. A method of treating a disease, illness, or condition comprising administering the composition of claim 7 as a primary therapeutic agent to a subject in need thereof.

15. The method of claim 14, further comprising administering an additional therapeutic agent or a diagnostic agent before, concurrently, or after administering the primary therapeutic agent.

16. The method of claim 15, wherein the additional therapeutic agent or diagnostic agent comprises a binding molecule, a drug, a prodrug, a toxin, an enzyme, an enzyme-inhibitor, a nuclease, a hormone, a hormone antagonist, an immunomodulator, a cytokine, an oligonucleotide, a chelator, a boron compound, a photoactive agent, a radionuclide, an anti-angiogenic agent, a dye, a radioopaque material, a contrast agent, a fluorescent compound, an enhancing agent, or combinations thereof.

17. The method of claim 16, wherein the binding molecule is multivalent.

18. The method of claim 16, wherein the binding molecule is multispecific.

19. The method of claim 15, wherein the binding molecule is an antibody.

20. The method of claim 15, wherein the additional therapeutic agent or diagnostic agent comprises an antibody conjugated to a drug or a toxin.

21. The method of claim 19, wherein the antibody comprises MAb 679, MAb 734, MAb Mu-9, MN-14, or combinations thereof.

22. The method of claim 16, wherein the binding molecule comprises a fusion protein.

23. The method of claim 14, wherein the disease, illness, or condition comprises a malignant disease, a cardiovascular disease, an infectious disease, an inflammatory disease an autoimmune disease, a metabolic disease, or a neurological disease.

24. The method of claim 23, wherein the disease, illness, or condition comprises a malignant disease and the binding molecule specifically binds a targeted tissue and wherein the targeted tissue comprises a tumor.

25. The method of claim 24, wherein the tumor produces or is associated with antigens selected from the group consisting of colon-specific antigen-p (CSAp), carcinoembryonic antigen (CEA), CD4, CD5, CD8, CD14, CD15, CD19, CD20, CD21, CD22, CD23, CD25, CD30, CD45, CD74, CD80, HLA-DR, Ia, Ii, MUC 1, MUC 2, MUC 3, MUC 4, NCA, EGFR, HER 2/neu, PAM-4, TAG-72, EGP-1, EGP-2, A3, KS-1, Le(y), S100, PSMA, PSA, tenascin, folate receptor, VEGF, PIGF, ILGF-1, necrosis antigens, IL-2, IL-6, TIOI, MAGE, and combinations thereof.

26. The method of claim 16, wherein the drug, prodrug, or toxin comprises aplidin, azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, semustine streptozocin, tamoxifen, taxanes, taxol, testosterone propionate, thalidomide, thioguanine, thiotepa, teniposide, topotecan, uracil mustard, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, Pseudomonas endotoxin, or combinations thereof.

27. The method of claim 16, wherein the radionuclide comprises ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Se, ⁷⁷As, ⁸⁶Y, ⁸⁹Sr, ⁸⁹Zr, ⁹⁰Y, ⁹⁴Tc, ^{94m}Tc, ⁹⁹Mo, ^{99m}Tc, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹ Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb ²¹²Bi, ²¹²Pb, ²¹³Bi, ²²³Ra, ²²⁵Ac, or mixtures thereof.

28. The method of claim 16, wherein the enzyme is selected from carboxylesterases, glucuronidases, carboxypeptidases, beta-lactamases, phosphatases, nucleases, proteases, lipases, and mixtures thereof.

29. The method of claim 16, wherein the immunomodulator or cytokine comprises IL-1, IL-2, IL-3, IL-6,1′-10, IL-12, IL-18, IL-21, interferon-α, interferon-β, interferon-γ, G-CSF, and GM-CSF, or mixtures thereof.

30. The method of claim 14, further comprising administering a diagnostic agent before, concurrently, or after administering the primary therapeutic agent.

31. The method of claim 30, wherein the diagnostic agent is a photosensitizer.

32. The method of claim 30, wherein the diagnostic agent comprises one or more image enhancing agents and the method further comprises performing magnetic resonance imaging (MRI).

33. A method of treating and/or diagnosing a disease or condition that may lead to a disease in a patient comprising:
(A) administering to the patient a binding molecule, wherein the binding molecule has at least one arm that binds a targeted tissue and at least one other arm that binds a targetable construct;
(B) optionally, administering to the patient a clearing composition and allowing the composition to clear non-localized binding molecules from circulation; and
(C) administering to the patient a targetable construct comprising the compound of claim 7.
